(19) United States
(12) Patent Application Publication
(10) Pub. No.: US 2009/0209650 A1
(43) Pub. Date: Aug. 20, 2009

(54) Materials and Methods for Improving the health of Shrimp

(76) Inventor: Francis Chi, Hong Kong (CN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/372,456

(22) Filed: Feb. 17, 2009

Related U.S. Application Data
(60) Provisional application No. 61/029,348, filed on Feb. 17, 2008.

(51) Int. Cl. A61K 31/145; A61P 37/00

(52) U.S. Cl. 514/665

(57) ABSTRACT

The subject invention provides methods for improving shellfish health. In specific embodiments, the invention provides methods for accelerating and/or augmenting shrimp growth; improving immunity; and enhancing fertility in shellfish. To do so, the present invention provides materials and methods for administering a cysteamine compound to shrimp.

### CROSS REFERENCE TO A RELATED APPLICATION

This application claims the benefit of U.S. provisional application Ser. No. 61/029,348, filed Feb. 17, 2008, herein by reference in its entirety.

### BACKGROUND OF THE INVENTION

The world's population is increasing rapidly, causing a concurrent increase in demand for seafood. Moreover, recent studies demonstrating the beneficial properties of shellfish have added to the demand (see Takezaki, T. et al., “Diet and lung cancer risk from a 14-year population-based prospective study in Japan: with special reference to fish consumption,” Nutr Cancer, 45(2):160-7 (2003); and Su, X. et al., “Omega-3 polyunsaturated fatty acid content in different edible portions of commercial scallop,” Asia Pac J Clin Nutr., 12 Suppl: S63 (2003)).

A variety of methods have been developed in attempts to bridge the gap between the supply and demand of seafood. For example, many different shellfish species are now produced in aquaculture. Some species are cultured during the complete life cycle whereas others are cultured from wild-harvested seed. Still others are raised in hatcheries and released to open water for later harvesting. Unfortunately, the cost in time and operation for producing marketable sized shellfish using current methods is very high.

The growth cycle for most shellfish is lengthy. Also, diseases and contaminated water are a threat to any shellfish aquaculture, hatchery, or farm. Most diseases in shellfish are caused by opportunistic pathogens, i.e., bacteria that cause disease in shellfish that are weakened or stressed. Microscopic marine organisms, such as the one-celled dinoflagellates that produce a toxin called paralytic shellfish poison (PSP), can accumulate in exposed shellfish and be harmful to the end-consumer.

Vaccines and antibiotics are two available methods used to protect shellfish against diseases. The use of drugs (i.e., antibiotics, vaccines, etc.), which is expensive, has to be considerably reduced in aquaculture to avoid environmental hazards and the risk of the development of resistance. Therefore, there is a constant need for enhancing the immunogenicity of shellfish.

### BRIEF SUMMARY OF THE INVENTION

The subject invention provides materials and methods for improving the health of shellfish. In particular, the present invention provides materials and methods for: accelerating and/or augmenting growth in shellfish; improving immunity to diseases and other contaminants; and/or increasing the success of larval production and survival.

The subject invention provides methods for improving the health of shellfish, such as shrimp, through the administration of a cysteamine compound to the shellfish. Specifically, an effective amount of a cysteamine compound is introduced to shellfish to promote shellfish health, growth, and population numbers. For example, cysteamine, or various cysteamine salts are administered to shellfish.

In a preferred embodiment, cysteamine is used to promote the health of shrimp. In an embodiment specifically exemplified herein, the shrimp are fed cysteamine in a cyclodextrin formulation. Specifically exemplified herein is the use of Aquanin Plus to improve shrimp health.

### BRIEF DESCRIPTION OF THE FIGURES

FIG. 1 is a graph that illustrates the quantity of enzyme phenoloxidase (Minute/Mg of Protein) of Vannamei after receiving different concentrations of Aquanin Plus for 0, 10, 20, 30, 40 and 50 days.

FIG. 2 is a graph that illustrates the quantity of blood cell (×10⁶ Cells/millilitre) of Vannamei after receiving different concentrations of Aquanin Plus for 0, 10, 20, 30, 40 and 50 days.

FIG. 3 is a graph that illustrates the percent of Vannamei white blood cells (phagocyte) to undergo phagocytosis of foreign pathogen upon receiving different concentrations of Aquanin Plus for 0, 10, 20, 30, 40 and 50 days.

FIG. 4 is a graph that illustrates concentration of Vannamei Serum decrease in Vibrio harveyi as compared with Control group upon receiving different concentrations of Aquanin Plus for 0, 10, 20, 30, 40 and 50 days.

FIG. 5 is a graph that illustrates the % Survival of Vannamei after receiving different concentrations of Aquanin Plus for 50 days after challenge with WSSV.

FIG. 6 is a graph that illustrates the % Survival of Vannamei after receiving different concentrations of Aquanin Plus for 60 days.

FIG. 7 is a graph that illustrates the differences in weight gain of Vannamei after receiving different concentrations of Aquanin Plus for 60 days.

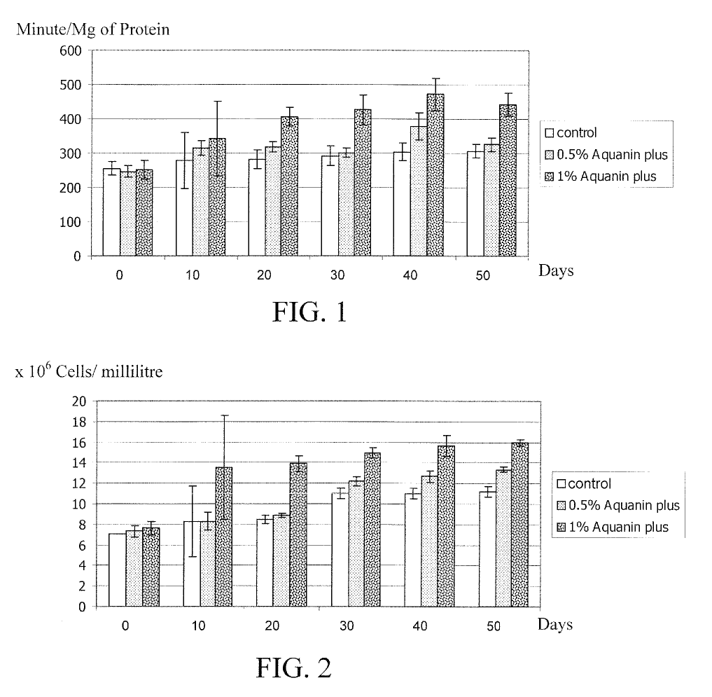

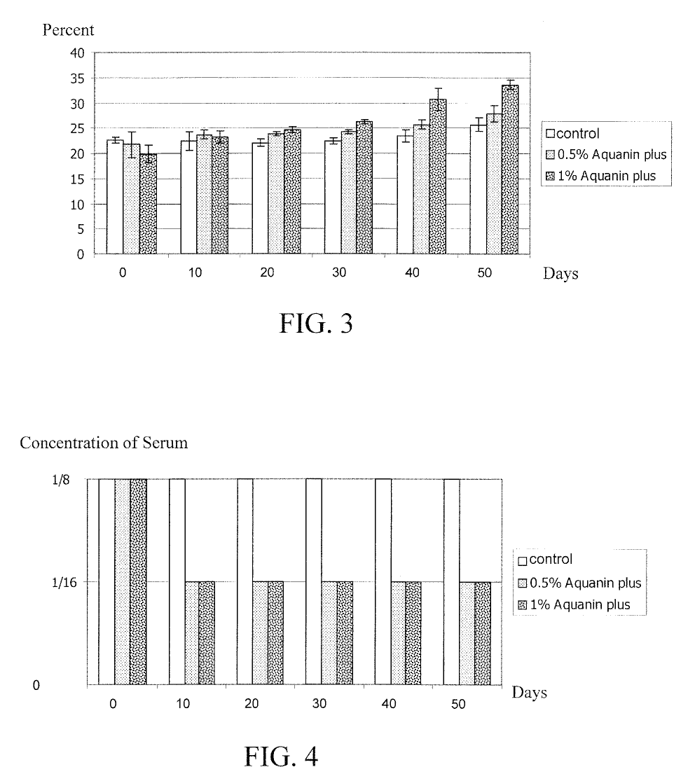

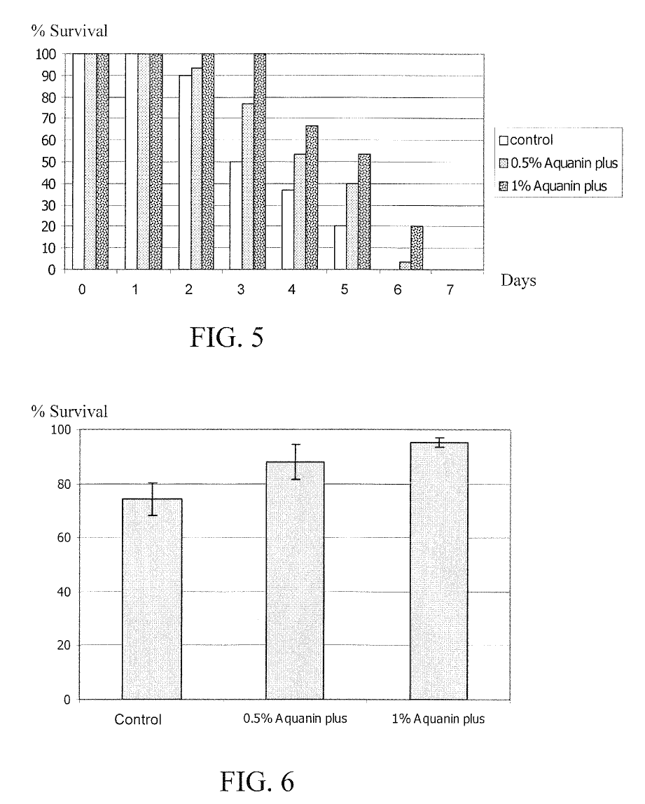

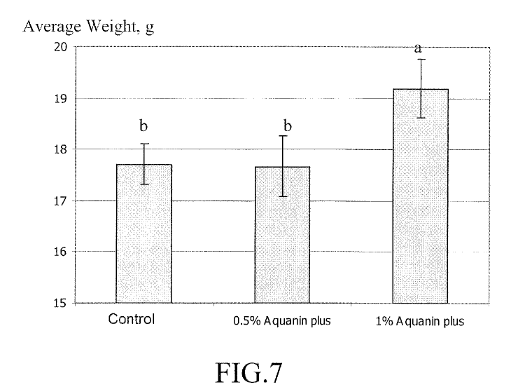

### DETAILED DISCLOSURE OF THE INVENTION

The subject invention provides unique methods for improving the health of shellfish. Specifically, the subject invention provides materials and methods for accelerating and/or augmenting growth in shellfish; improving immunity to diseases and other contaminants; and/or increasing the success of larval production and survival.

In particular, the invention concerns administering a cysteamine compound to shellfish, in an amount effective to promote shellfish health, growth, and population numbers. One embodiment of the invention involves the use of a composition for improving shellfish health, wherein the composition comprises a cysteamine compound. Specifically exemplified herein is the use of cysteamine in a cyclodextrin formulation. Preferably, β-cyclodextrin cysteamine hydrochloride is used to promote the health of shrimp.

As contemplated herein, shellfish includes aquatic invertebrates having a soft, unsegmented body that can be enclosed in a shell. For example, references to shellfish include crustaceans such as prawns, shrimp, crawfish, crayfish, crabs, lobsters; and mollusks such as abalone, clams, mussels, oysters, scallops, octopi, squid, and snails. References to shellfish herein can also include turtles, sea urchins, and sea cucumbers.

The term “shellfish health,” as used herein, generally refers to a variety of parameters that affect the overall condition of a shellfish. Specific parameters upon which shellfish health is based include: the size (or growth) of a shellfish; the length of time in a growing cycle; the immune system's ability to adequately address exposure to diseases and contamination; and the ability to reproduce offspring. As contemplated herein, improving shellfish health can include reducing shellfish mortality; increasing antibody titer/lymphocyte number; and/or increasing cytokine secretion.

“Concurrent administration” and “concurrently administering,” as used herein, includes administering a compound or method suitable for use with the methods of the invention (administration of a cysteamine compound) to improve shellfish health. For example, a vitamin, an antibiotic and/or vaccine can be administered concurrently with the materials and methods of the invention to improve shellfish health.

According to the subject invention, a compound can be provided in admixture with a cysteamine compound, such as in an aqueous emulsion; or the compound and cysteamine can be provided as separate compounds, such as, for example, separate compositions administered consecutively, simultaneously, or at different times. Preferably, if the cysteamine compound and the known agent (or therapeutic method) for improving shellfish health are administered separately, they are not administered so distant in time from each other that the cysteamine compound and the known agent (method) cannot interact.

As used herein, reference to a “cysteamine compound” includes cysteamine and cysteamine salts. Contemplated cysteamine salts include, but are not limited to, cysteamine hydrochloride, cysteamine chlorohydrate, cysteamine acetate, cysteamine phosphate, cysteamine nitrate, cysteamine bromide, cysteamine bitartrate, and cysteamine fluoride. Also included within the scope of the subject invention are analogs, derivatives, conjugates, and metabolites of cysteamine, which have the ability as, described herein to improve shellfish health. Various analogs, derivatives, conjugates, and metabolites of cysteamine are well known and readily used by those skilled in the art and include, for example, compounds, compositions and methods of delivery as set forth in U.S. Pat. Nos. 6,521,266; 6,468,522; 5,714,519; and 5,554,655.

The term “effective amount,” as used herein, refers to the amount necessary to elicit the desired biological response. In accordance with the subject invention, the effective amount of a cysteamine compound is the amount necessary to improve shellfish health. In a preferred embodiment, the effective amount of a cysteamine compound is the amount necessary to accelerate and/or augment growth in shellfish; improve immune response to diseases and other contaminants; enhance fertility; and/or increase the success of larval production and survival. For example, the improvement in shellfish health can be a 1%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, or 300% acceleration and/or augmentation in growth; improvement in immunity response to a disease and/or contaminant; and/or enhancement in fertility. More specifically, shellfish health is improved as a result of reduced shellfish mortality; increased antibody titer/lymphocyte numbers; and increased cytokine secretion.

In certain embodiments, the cysteamine compound is cysteamine hydrochloride and the effective amount in a composition is between about 20-40% of the composition. In other embodiments, cysteamine hydrochloride is present in the composition in amounts greater than or equal to 0.01%, 0.05%, 1%, 2%, 5%, 10%, 15%, 20%, 25%, 30%, 35%, 40%, or more than 40% of the composition. In yet other embodiments, cysteamine hydrochloride is present in the composition in amounts greater than or equal to 15 g, 20 g, 25 g, or 30 g per kg of the composition. In other embodiments, cysteamine hydrochloride is present in the composition in amounts greater than or equal to 200 g, 225 g, 250 g, 275 g or 300 g per kg of the composition. In further embodiments, cysteamine can be present at about 150 g to about 1,000 g per ton of feed, more preferably between about 200 g and 900 g per ton of feed, and even more preferably between 250 g and 500 g per ton of feed. In yet another embodiment, cysteamine hydrochloride is present in the composition at about 270 g per ton of feed.

Accelerated and/or Augmented Growth

In one embodiment of the invention, a cysteamine compound is administered to shellfish to accelerate and/or augment growth. As contemplated herein, to “accelerate and/or augment growth” refers to the ability to shorten developmental periods during a normal growth cycle and/or increase the overall size of the shellfish. In a preferred embodiment, cysteamine hydrochloride is administered to shrimp to accelerate and/or augment growth.

### Improving Immune Response

In another embodiment, a cysteamine compound is administered to shellfish to improve immunity response to diseases and other contaminants. As contemplated herein, “improving immunity” refers to boosting the shellfish immune system to more effectively attack harmful microorganisms and/or contaminants and heal the shellfish of any damages incurred by exposure to such microorganisms and/or contaminants. By improving immunity, the subject invention also increases the likelihood of success in larval production and survival. In a preferred embodiment, a cysteamine compound is administered to shrimp to improve immunity response to diseases and other contaminants.

According to the subject invention, introduction of a cysteamine compound to shellfish, such as shrimp, can: (1) proactively augment shellfish immunity to promote resistance to disease and/or contamination; and/or (2) treat and promote rapid recovery from current exposure to harmful microorganisms and/or contamination.

As contemplated herein, the subject invention improves shellfish immune response to a variety of microorganisms and contaminants. For example, the subject invention improves oyster immune response to a variety of diseases and pathogens including, without limitation, Oyster Velar Virus Disease (OVVD); Gill Disease of Portuguese Oysters; Haemocytic Infection Virus Disease of Oysters; Herpes-Type Virus Disease of Oysters; Extracellular Giant “Rickettsiae” of Oysters; Perkinsus marinus (“Dermo” Disease) of Oysters; Perkinsus sp. of European Flat Oysters; Kidney Coccidia of Oysters; Minchinia armoricana of Oysters; Marteiliosis (Aber disease) of Oysters; Marteilioides chungmuensis of Oysters; Bonamia ostreae of Oysters; Oyster Egg Disease; Invasive Ciliates of Juvenile Oysters: Mytilicola intestinalis (Red Worm Disease) of Oysters; Haemocytic Neoplasia of Oysters; Juvenile Disease of Eastern Oysters; Viral Gametocytic Hypertrophy of Oysters; Nocardiosis of Oysters; Mikrocytos machine (Denman Island Disease) of Oysters; Haplosporidium nelsoni (MSX) of Oysters; Haplosporidium costale (SSO) of Oysters; Ostracoblabe implexa (Shell Disease) of Oysters; Oyster Trematode Diseases; Mytilicola orientalis (Red Worm) of Oysters; Parasite Copepods on Oyster Gills; Pea Crabs in Oysters; Malpeque Disease of Oysters; Papova-Like Virus Infection of Pearl Oysters; Apicomplexan Parasite of New Zealand Oysters; Haplosporidium sp. of Pearl Oysters; Marteilia sydneyi of Oysters; Marteilioides branchialis of Oysters; Bonamia exitiosus (Bonamiasis of New Zealand Dredge Oysters); Bonamia sp. (Bonamiasis of Australian Oysters); Mikrocytos roughleyi (Australian Winter Disease) of Oysters; Microsporidiosis of Dredge Oysters; Rikettsia-like and Chlamydia-like Organisms of Oysters; Vibrio spp. (Larval and Juvenile Vibriosis) of Oysters; Hinge Ligament Disease of Juvenile Oysters; Digestive Tract Impaction of Larval Oysters; Gregarine Parasitism of Oysters; Hexamitiasis of Oysters; Ancistrocoma-like Ciliates of Oysters; Sphenophyra-like Ciliates of Oysters; Gill Trichodinids of Oysters; Sirolpidium zoophthorum (Larval Mycosis) of Oysters; Oyster Gill Turbellaria; Nematode Parasitism of Oysters; Shell-boring Polychaetes of Oysters; Shell-burroing Sponges of Oysters; and Pyramidellid Snails of Oysters.

In another example, the subject invention can improve mussel immune response to a variety of diseases and pathogens including, without limitation, Virus-like Diseases of Mussels; Haplosporidian Infection of Mussels; Marteilia refringens/maurini of Mussels; Steinhausia mytilovum (Mussel Egg Disease); Phototrophic Endolity Invasion of Mussel Shells; Proctoeces maculates Trematode Disease of Mussels, Mussel Gill Turbellaria; Mytilicola intestinalis (Red Worm Disease) of Mussels; Kidney Coccidia of Mussels; Bucephalid Trematode Diseases of Mussels; Mytilicola orientalis (Red Worm) of Mussels; Pea Crabs in Mussels; Haemocytic Neoplasia of Mussels; Mytilicola porrecta (Red Worm) of Mussels; Rickettsia-like and Chlamydia-like Organisms of Mussels; Gregarine Parasitism of Mussels; Intracellular Ciliates of Mussels; Phenophyra-like Ciliates of Mussels; Ancistrum mytili Gill Ciliate of Mussels; Mycotic Periostracal Sloughing of Mussels; Trematode Metacercariae of Mussels; Parasitic copepods on Mussel Gills; Shell-boring Polychaetes of Mussels; and Shell-burrowing Sponges of Mussels.

In yet another example, the subject invention can improve clam and cockle immune response to a variety of diseases and pathogens including, without limitation, Viral infections of Clams; Brown Ring Disease of Manila Clams; Perkinsus of Clams and Cockles; Haplosporidian Infection of Clams; Microsporidiosis of Clams; Amoeboflagellate Disease of Larval Geoduck Clams; Mytilicola intestinalis (Red Worm Disease) of Clams and Cockles; Nuclear Inclusion X (NIX) of Pacific Razor Clams; Kidney Coccidia of Clams; QPX (quahog parasite unknown) of Clams; Mytilicola orientalis (Red Worm) of Clams and Cockles; Pea Crabs in Clams and Cockles; Haemocytic Neoplasia of Clams; Gonadal Neoplasia of Clams; Endonucleobiotic Bacteria of Clams in Portugal; Mycoplasma-like Infection of Cockles; Cryptosporidiosis of Clams; Marteilia-like Parasite of Giant Clams; Amoebiasis of Cockles; Rickettsia-like and Chlamydia-like Organisms of Clams and Cockles; Vibrio spp. (Larval and Juvenile Vibriosis) of Clams; Hinge Ligament Disease of Juvenile Clams; Gregarine Parasitism of Clams and Cockles; Sphenophyra-like Ciliates of Clams and Cockles, Ancistrocoma pelseneeri and A. myae Ciliates of Clams; Gill Trichodina of Clams and Cockles; Sirolpidium zoophthorum (Larval Mycosis) of Clams; Turbellaria of Clams, Trematode Metacercariae of Clams and Cockles; Shell-boring Polychaetes of Clams; and Siphon Snails of Clams and Cockles.

In a further example, the subject invention can improve scallop immune response to a variety of diseases and pathogens including, without limitation, Perkinsus sp. of Japanese Scallops in Asia; Scallop Haplosporidian; Marteilia sp. of Scallops; Brood-;pouch Copepod of Scallop Gills; Pea Crabs in Scallops, Intracellular Bacterial Disease of Scallops; Bacterial Abscess Lesions of Scallops; Perkinsus qugwadi (SPX) of Scallops; Kidney Coccidia of Scallops; Perdinsus karlssoni of Scallops; Scallop Protistan G; Microsporidiosis of Scallops; Trematode Metacercariae of Scallops; Virus-like Infection of Scallops; Chlamydiosis of Scallops; Rickettsia-like and Chlamydia-like Organisms of Scallops; Vibrio spp. (Larval Vibriosis) of Scallops; Gregarine Parasitism of Scallops; Gill Trichodinids of Scallops; Scallop Gill Turbellaria; Nematode Parasitism of Scallops; Shell-boring Polychaetes of Scallops; and Shell-burrowing Sponges of Scallops.

In another example, the subject invention can improve abalone immune response to a variety of diseases and pathogens including, without limitation, Kidney Coccidia of Abalone; Sabellid Polychaete Infestation of Disease in Abalone; Labyrinthuloides haliotidis of Abalone; Amyotrophia of Abalone; Blister Disease of Cultured Abalone; Withering Syndrome of Abalone; Perkinsus olseni of Abalone; Haplosporidian parasite of Abalone; Fungal Disease of Abalone; Nematode Parasitism of Abalone; Bacterial Diseases of Abalone; Ciliates Associated with Abalone; Trematode Metacercariae of Abalone; and Shell-borrowing Polychaetes of Abalone.

In yet another example, the subject invention can improve sea urchin immune response to a variety of diseases and pathogens including, without limitation. Namatode Parasitism of Sea Urchin; Bald-Sea-Urchin Disease; Paramoeba invadens of Sea Urchins; Spotted gonad Disease of Sea Urchins; Black Sea Urchin Plague; Trematode Metacercariae of Sea Urchins; and Turbellarian Parasitism of Sea Urchins.

In yet another example, the subject invention can improve lobster immune response to a variety of diseases and pathogens including, without limitation, Paramoeba perniciosa (Paramoebiasis) of Lobsters; Gaffkemia of Lobsters: Anophryoides haemophila (Ciliate Disease) of Lobsters; Pseudocarcinonemetes homari of Lobsters; Microsporidosis of Lobsters; Hematodinium sp. of Norway Lobster; Carcinonemertes australiensis of Lobsters; Parasitic Copepods of Lobsters; vibrio spp. (Juvenile Vibriosis) of Lobsters; Chitinolytic Bacterial Shell Disease of Lobsters; Gregarine Parasitism of Lobsters; Lagenidium sp. (Fungus Disease) of Lobsters; Fusarium sp. (Fungus or Burn Spot Disease) of Lobsters; Haliphthoros sp. (Fungus Disease) of Lobsters. Nematodes in Lobsters; Trematode Metacercariae in Lobsters; and Acanthocephalan Larvae in Lobsters.

In yet another example, the subject invention can improve shrimp and prawn immune response to a variety of diseases and pathogens including, without limitation, Rickettsia-like Infection of Pandalid Shrimp; Protistan Pathogen of Pandalid Shrimp (SPP); Sylon (Rhizocephalan Disease) of Shrimp and Prawns; Baculovirus penaei (BP Virus Disease) of Penaeid Shrimp; Monodon Baculovirus (MBV) Disease of Penaeid Shrimp; Baculoviral Midgut-gland Necrosis (BMN) of Penaeid Shrimp; White Spot Syndrome Baculovirus Complex of Penaeid Shrimp; Hepatopancreatic Parvovirus (HPV) Disease of Shrimp and Prawns; Infectious Hypodermal and Haematopoietic Necrosis Virus (IHHNV) of Penaeid Shrimp; Lymphoidal Parvo-like Virus Disease of Penaeid Shrimp; Lymphoid Organ Vacuolization Virus (LOVV) of Penaeid Shrimp; Reo-like Virus (REO) Disease of Penaeid Shrimp; Taura Syndrome Virus of Penaeid Shrimp; Rhabdovirus Disease of American Penaeid Shrimp; Yellow-head Virus Disease (YHD) of Penaeid Shrimp; Rickettsial Infection of Penaeid Shrimp; Necrotizing Hepatopancreatic of Penaeid Shrimp; Mycobacteriosis of Penaeid Shrimp; Vibrio penaeicida of Cultured Kurama Prawns; Larval Bacterial Necrosis of Freshwater Shrimp; Haplosporidian Infections of Penaeid Shrimp; Gregarine Disease of Penaeid Shrimp; Ciliate Disease of Penacid Shrimp; Gut and Nerve Syndrome (GNS) of Penaeid Shrimp; Larval Mid-cycle Disease (MCD) of Freshwater Shrimp; Red Disease of Penaeid Shrimp; Vibrio spp. (Vibrio Disease) of Cultured Shrimp; Chitinolytic Bacterial Shell Disease of Shrimp and Prawns; Filamentous Bacterial Disease of Shrimp and Prawns; Microsporidosis (Cotton Shrimp Disease) of Shrimp and Prawns; Larval Mycosis of Shrimp and Prawns; Fusarium sp. (Fungus Disease) of Shrimp and Prawns; Nematomorph Parasitism of Pandalid Shrimp; and Black Gill Syndrome of Shrimp and Prawns.

In yet another example, the subject invention can improve crab immune response to a variety of diseases and pathogens including, without limitation, Viral Diseases of Crabs; Rickettsia and Chlamydia of Crabs; Haplosporidosis of Crabs; Paramoeba perniciosa (Grey Crab Disease); Hematodinium perezi and Hematodinium sp. of Atlantic Crabs; Chitinolytic Fungal Disease (Black Mat Syndrome) of Crabs; Carcinonemertes spp. of Crabs; Mesanophrys spp. (Ciliate Disease) of Crabs; Hematodinium sp. (Bitter Crab Disease); Rhizocephalan Parasites of Crabs; Hematodinium spp. of Crabs in Australia; Chitinolytic Bacterial Shell Disease of Crabs; Microsporidosis of Crabs; Trematode Metacercariae of Crabs; Acanthocephalan Parasitism of Crabs; Nematomorph Parasitism of Crabs; and Lagendium spp. (Fungus Disease) of Crabs.

In yet another example, the subject invention can improve crayfish immune response to a variety of diseases and pathogens including, without limitation, Psorospermium spp. (Protozoan Disease) of European Crayfish; Therlohaniasis of Crayfish; Burn Spot Disease (Fungus Disease) of Crayfish; Crayfish Plague (Fungus Disease); Baculovirus of Blue Crayfish; Rickettsia of Crayfish; Nocardia sp. (Bacterial Disease) Crayfish; Proteus or Pseudomonas Bacterial Septicaemia of Crayfish; Chitinolytic Bacterial Shell Disease of Crayfish; Psorospermium sp. (Protozoan Infection) of American Crayfish; Saprolegnia spp. (Fungus Disease) of Crayfish; Trematodes in Crayfish; Turbellaria Infestation of Crayfish; and Branchiobdellida Annelid Parasitism of Crayfish.

Administration of a cysteamine compound to shellfish, in accordance with the subject invention, can be accomplished by any suitable method and technique presently or prospectively known to those skilled in the art. Specifically exemplified herein is the introduction of a cysteamine compound, either alone or concurrently with additional compound(s) or method(s), into water containing the shellfish to be treated. The cysteamine compound can be introduced as a composition, in any available form including in a liquid (i.e., solvent, oil), in an aqueous mixture, in an aqueous emulsion, in a solid carrier or substrate, or other vehicles provided the vehicles are compatible with the administration of the cysteamine compound into water harboring the shellfish to be treated, and do not adversely affect the shellfish.

A variety of suitable adjuvants may also be used in compositions comprising a cysteamine compound. For example, emulsifiers, antifoaming agents (or defoaming agents), antioxidants, vitamins, minerals, preservatives, coloring agents, and the like can be included in compositions of the invention. In one embodiment, the adjuvants are present in compositions of the invention in minor amounts, i.e. less than about 5% by volume, and preferably, less than 1% by volume. In other embodiments, greater amounts of adjuvants are present in compositions of the invention, i.e., up to 70% by volume. All such adjuvants should be noninjurious and nontoxic to shellfish being treated.

In certain embodiments, vitamins are present in compositions of the invention. In a preferred embodiment, vitamin E is present in a composition (prior to addition of any feed) between about 0.05% to 1.0%. In another embodiment, vitamin E is present in the composition in amounts greater than or equal to 0.1%. In yet another embodiment of the invention, vitamin E is present in the composition in amounts greater than or equal to 0.1 mg per kg of the composition. In a further embodiment, where 1 ton of feed is added to the composition, the vitamin E is present in the composition in amounts greater than or equal to 10 mg per ton of feed.

According to the present invention, suitable emulsifiers (i.e., surfactants or dispersants) can be cationic, anionic, nonionic, or amphoteric emulsifiers. Preferred emulsifiers include, for example, food grade emulsifiers which are widely available. An overview of some types of suitable emulsifiers for use with the invention include those set forth in A. J. St. Angelo, “A Brief Introduction to Food Emulsion and Emulsifiers,” at pp. 1-8 of G. Charalambous et al., Eds., Food Emulsifiers-Chemistry, Technology, Functional Properties and Applications, Elsevier Science Publishing Co. Inc., New York, N.Y. (1989).

In one embodiment, compositions comprising a cysteamine compound and a carrier such as inclusion compound host materials are provided. It is believed that by providing a carrier such as inclusion compound host materials, a stabilized cysteamine compound molecule can be safely delivered to shellfish that will not induce toxicity. In addition, such carrier materials can include coating materials (i.e., enteric-coatings) that allow dissolution of the coating in an alkaline environment such as in the intestines. Examples of carrier materials for use in accordance with the subject invention include, but are not limited to, inclusion compound host materials, microcrystalline cellulose, starches, and sodium alginate.

In certain embodiments, the carrier materials present in compositions of the invention comprise: microcrystalline cellulose present between about 10-30 wt %; starch present between about 40-50 wt %; and sodium alginate present between about 1-5 wt %. In a related embodiment, the microcrystalline cellulose is present in the composition at amounts greater than or equal to 20%, the starch is present in the composition at amounts greater than or equal to 43%, and the sodium alginate is present in the composition at amounts greater than or equal to 3.5%. In yet another related embodiment of the invention, the microcrystalline cellulose is present in the composition (prior to addition of feed to the composition) in amounts greater than or equal to 20.0 g per kg of the composition; the starch is present in amounts greater than or equal to 43.0 g per kg of the composition; and the sodium alginate is present in amounts greater than or equal to 3.5 g per kg of the composition. In a further embodiment, where one ton of feed is added to the composition, the microcrystalline cellulose is present in amounts greater than or equal to 200 g per ton of feed; the starch is present in the composition in amounts greater than or equal to 430 g per ton of feed; and the sodium alginate is present in amounts greater than or equal to 35 g per ton of feed.

An inclusion compound host material that can be used in accordance with the subject invention include those disclosed in U.S. patent application Ser. No. 20040033985, incorporated herein in its entirety. Contemplated inclusion compound host materials include proteins (such as albumin), crown ethers, polyoxyalkylenes, polysiloxanes, zeolites, cholestyramine, colestipol, colesevelam, colestimide, sevelamer, cellulose derivatives, dextran derivatives, starch, starch derivatives, and pharmaceutically acceptable salts thereof. Contemplated cellulose derivatives and dextran derivatives include DEAE-cellulose, guanidinoethylcellulose, or DEAE-Sephadex. Favorable starches or starch derivatives to be included in the compositions of the invention include cyclodextrin, retrograded starch, degraded starch, a combination of retrograded and degraded starch, hydrophobic starch, amylase, starch-diethylaminoethylether, and starch-2-hydroxyethylether.

According to the subject invention, preferred inclusion compound host materials include, but are not limited to, cyclodextrin and/or its derivatives (i.e., methyl β-cyclodextrin, hydropropyl β-cyclodextrin, hydroethyl β-cyclodextrin, polycyclodextrin, ethyl β-cyclodextrin and branched cyclodextrin. As one skilled in the art will appreciate, any cyclodextrin or mixture of cyclodextrins, cyclodextrin polymers, or modified cyclodextrins can be utilized pursuant to the present invention. For example, β-cyclodextrins are widely used as solubilizing agents, stabilizers, and inert excipients in pharmaceutical compositions (see U.S. Pat. Nos. 6,194,430; 6,194,395; and 6,191,137, each of which is incorporated herein by reference in its entirety). Cyclodextrins are available from Wacker Biochem Inc., Adrian, Mich. or Cerestar USA, Hammond, Ind., as well as other vendors.

The general chemical formula of cyclodextrin is (C₆O₅H₉)ₙ. β-cyclodextrins are cyclic compounds containing seven units of α-(1,4) linked D-glucopyranose units, and act as complexing agents that can form inclusion complexes and have concomitant solubilizing properties (see U.S. Pat. No. 6,194,395; see, also, Szeitli, J. Cyclodextrin Technol, 1988). Formation of inclusion complexes using cyclodextrin or its derivatives protects the constituent (i.e., cysteamine compound) from loss of evaporation, from attack by oxygen, acids, visible and ultraviolet light and from intra- and intermolecular reactions. The content of inclusion compound host materials in compositions of the subject invention can range from about 1 to 80 wt %. Preferably, the content of inclusion compound host materials in compositions of the invention range from about 1 to 60 wt %. The actual amount of the inclusion compound host materials used will depend largely upon the actual content of cysteamine compound and additional therapeutic agent(s) used in preparing compositions of the invention.

In certain embodiments of the invention, the inclusion compound host material is β-cyclodextrin and it is present between about 1% to 20% of the composition prior to addition of feed. In another embodiment, the β-cyclodextrin is present in the composition in amounts greater than or equal to 5%. In yet another embodiment of the invention, β-cyclodextrin is present in the composition in amounts greater than or equal to 5.0 g per kg of the composition. In a further embodiment where one ton of feed is added to the composition, the β-cyclodextrin is present in the composition in amounts greater than or equal to 50 g per ton of feed.

Where needed, after the introduction of a cysteamine compound to water in which shellfish are harbored, a metering or mixing pump, or an inline mixer (i.e., a mixing valve, nozzle or orifice), an aerator, or other device known to the skilled artisan may be used to accomplish the direct dispersion of the cysteamine compound in water.

In one embodiment, an aqueous mixture, emulsion, or dispersion including a cysteamine compound is introduced into water harboring shellfish to be treated. The aqueous mixture, emulsion, or dispersion of the invention can contain from about 0.1% to about 95% of a cysteamine compound, wherein all percentages being by volume, based on the final volume of the composition. The composition can be further diluted when added to the water environment containing the shellfish to be treated according to the present invention. The amount of cysteamine compound used can be varied based upon the health (i.e., size, age, etc.) of the shellfish to be treated.

According to the subject invention, a composition comprising a cysteamine compound can be concurrently administered with shellfish feed. In one embodiment, the composition comprising a cysteamine compound is mixed with shellfish feed prior to feeding the shellfish. In a preferred embodiment, a composition comprising β-cyclodextrin cysteamine hydrochloride is mixed with a solid feed mixture (i.e., sinking or floating feed) and is introduced to shrimp to be treated. The feed mixture of the invention can contain from about 0.1% to about 95% of a cysteamine compound, wherein all percentages being by volume, based on the final volume of the composition. The amount of cysteamine compound used can be varied based upon the health (i.e. size, age, etc.) of the shellfish to be treated.

According to the subject invention, compositions of the invention further comprise shellfish feed. In certain embodiments of the invention, a composition comprising a cysteamine compound, a carrier, and vitamin E are mixed with shrimp feed to treat shrimp. In related embodiments, the shellfish feed is about 1 ton of shrimp feed. In further embodiments, the composition comprises about 200-400 g of a cysteamine compound (such as cysteamine hydrochloride) per ton of shrimp feed. In further embodiments of the invention, the composition comprises about 270 g of a cysteamine compound (such as cysteamine hydrochloride) per ton of shrimp feed. In yet another embodiment, about 350-450 μg of a composition comprising about 20-30% cysteamine compound, about 1-20% cyclodextrin, and about 0.05-1.0% vitamin E is administered to an average shrimp per day, where the average shrimp size is about 12 g.

The cysteamine compounds of the subject invention can be formulated according to known methods for preparing compositions for use in administration to shellfish. The formulations may be presented in unit-dose or multi-dose containers, for example sealed ampoules and vials, and may be stored in a freeze dried (lyophilized) condition requiring only the condition of the sterile liquid carrier, for example, water, prior to use. Extemporaneous solutions and suspensions may be prepared from sterile powder, granules, tablets, etc. It should be understood that in addition to the ingredients particularly mentioned above, the formulations of the subject invention can include other agents conventional in the art having regard to the type of formulation in question.

In certain methods of the invention, the effective amount of cysteamine introduced is dependent on factors such as water pH, hardness, alkalinity, temperature, and the like. According to one embodiment of the subject invention, an effective amount of cysteamine for administration is between about 1-20 μg of a cysteamine salt (such as cysteamine hydrochloride) per gram of shellfish per day. In a related embodiment, about 1-20 μg cysteamine hydrochloride is administered per gram of shrimp per day. In yet another related embodiment, about 3-15 μg cysteamine hydrochloride is administered per gram of shrimp per day. In yet another related embodiment, about 5-10 μg cysteamine hydrochloride is administered per gram of shrimp per day.

The shellfish that are treated according to the invention include those that are held in a confined body of water, such as a shipping container, holding tank, aquarium, pool, or small pond, large body of water and those that are round in unconfined water, such as streams or off of a beach.

Following are examples that illustrate procedures for practicing the invention. These examples should not be construed as limiting. All percentages are by weight and all solvent mixture proportions are by volume unless otherwise noted.

### Experimental Examples

Currently, Pacific white shrimp, Litopenaeus vannamei, native of Central and South America, is the major shrimp species cultured in Thailand, Taiwan, and China. Since 2001, shrimp farmers have experienced disease problems associated with production declines in farmed L. vannamei.

Aquanin Plus (trade name) from Walcom Bio-Chemicals Industrial Limited, contains at least 27% β-cyclodextrin cysteamine hydrochloride and 0.1% Vitamin E. For the purpose of the following experiments, 270 g of cysteamine was present in 1 kg of Aquanin Plus. In an embodiment, 300 ppm tip to 4,000 ppm of Aquanin plus is effective in treating shrimp in accordance with the subject invention. The following experiments provide insight into concentrations of Aquanin Plus useful in increasing growth, survival and immune response for use in shrimp culture.

### Experiment 1

L. vannamei (11.6+0.5 g) were obtained from a commercial farm in Chantaburi province, Thailand and acclimated in the laboratory for two weeks before experimentation. For the determination of the growth-promoting and immunostimulant effects of Aquanin Plus administration in the diet, tests were carried out in three treatments (with six replicates/treatment). Each replicate consisted of 25 shrimp in 500-liter tanks. Shrimp were fed four times daily at 3% body weight per day for 50 days with pelleted feed containing graded levels of Aquanin Plus (0%, 0.05% and 0.1% of the feed; for example, 0.1% Aquanin Plus refers to 1 kg of Aquanin Plus added to 1 ton feed and 0.05% Aquanin Plus refers to 0.5 kg Aquanin Plus added to 1 ton feed). During the experiment, water quality parameters were maintained as follows: temperature at 28+1° C., pH at 7.8-8.0 and salinity at 25 ppt. The data were subjected to one-way analysis of variance followed by Duncan's multiple range test.

After 50 days of dietary administration, shrimp fed with 0.1% Aquanin Plus had a percentage increase in body weight significantly higher (P<0.05) than the Aquanin Plus 0.05% and control groups (Table 1).

TABLE 1
Percentage weight increase and survival of L. vannamei after
50 days of feeding with Aquanin Plus at 0%, 0.05% and 0.1%.

| | Control | Aquanin Plus | |
|---|---|---|---|
| Treatment | 0% | 0.05% | 0.1% |
| % weight increase | 52.69 ± 3.45^{a} | 52.29 ± 5.05^{a} | 65.45 ± 4.84^{b} |
| % survival | 74.40 ± 6.07^{a} | 88.00 ± 6.32^{b} | 95.20 ± 1.79^{b} |

The results showed that shrimp which fed on diets containing 0.1% Aquanin Plus had significantly higher (P<0.05) total haemocyte count (THC), percentage phagocytosis and phenoloxidase activity than the 0.05% Aquanin Plus and control groups (Table 2).

TABLE 2
Total haemocyte count (THC), percentage phagocytosis, phenoloxidase
activity and bacterial activity of L. vannamei after 50 days
of feeding with different levels of Aquanin Plus.

| | Control | Aquanin Plus | |
|---|---|---|---|
| | 0% | 0.05% | 0.1% |
| THC (×10⁶ | 11.13 ± 0.51^{a} | 13.38 ± 0.29^{a} | 15.98 ± 0.29^{b} |
| cell/ml) | | | |
| % phagocytosis | 25.75 ± 1.34^{a} | 27.86 ± 1.64^{a} | 33.61 ± 0.85^{b} |
| Phenoloxidase | 307.08 ± 18.92^{a} | 326.65 ± 19.98^{a} | 441.98 ± 32.63^{b} |
| activity | | | |
| (unit/min/mg. | | | |
| protein) | | | |
| Bactericidal | 1:8 | 1:16 | 1:16 |
| activity | | | |

In conclusion, oral administration of 0.1% Aquanin Plus for 50 days increases the growth, survival and immune response of L. vannamei.

### Experiment 2

Litopenaeus vannamei (11-12 g) were obtained from a commercial shrimp farm in Chantaburi province, Thailand. A total of 1,500 farm-reared shrimp were transported and acclimated in fiberglass tanks. After 14 days of acclimatization, shrimp were used for the experiment. Salinity during the acclimation period and experiment was maintained at 25 ppt.

For the determination of the growth-promoting and survival effects of Aquanin Plus administration in the diet, tests were carried out in three treatments that consisted of 0.05% Aquanin Plus, 0.1% Aquanin Plus and control feed (with six replicates/treatment). Each replicate consisted of 25 shrimp of (11-12 g) in 500-liter tanks. Shrimp were fed four times daily at 3% body weight per day for 60 days with pelleted feed containing graded levels of Aquanin Plus (0%, 0.05% and 0.1% of the feed). Feeding rate was adjusted according to shrimp weight throughout the 60-day experiment period. Water quality parameters such as pH, dissolved oxygen (DO), ammonia, nitrite were measured weekly throughout the experiment. The growth and survival rates of all treatment groups were recorded every 20 days for 60 days.

Shrimp from Experiment 2 (60 shrimp/treatment) were challenged with white spot syndrome virus (WSSV) by single feeding fed with WSSV infected shrimp at 12% of body weight. Number of dead shrimp were recorded for 7 days.

Shrimp from Experiment 2 (60 shrimp/treatment) were also challenged with yellow-head virus (YHV) by feeding with YHV infected shrimp at 12% of body weight one time. Number of dead shrimp were recorded for 7 days.

Data were subjected to one-way analysis of variance followed by Duncan's multiple range test. Differences were considered significant if P<0.05.

### Results—Determination of the Aquanin Plus Effect on the Growth and Survival of White Shrimp

After 40 days of administration in diet, shrimp fed with 0.1% Aquanin Plus had an average body weight that was significantly higher (P<0.05) than that of Aquanin Plus at 0.05% and the control (Table 3). Considering the percentages survival, there was no significant difference between Aquanin Plus-treated shrimps but they were significantly higher (P<0.05) than those of the control group (Table 4).

TABLE 3
Average body weight of L. vannamei after 60 days of
feeding with Aquanin Plus at 0, 0.05% and 0.1%.

| Feeding | Average body weight (g) | | |
|---|---|---|---|
| period | | 0.05% Aquanin | 0.1% Aquanin |
| (day) | control | Plus | Plus |
| 0 | 11.76 ± 0.42a | 11.78 ± 0.46a | 11.86 ± 0.48a |
| 20 | 12.28 ± 0.62a | 12.73 ± 0.78ab | 12.97 ± 0.78b |
| 40 | 15.40 ± 1.19a | 15.84 ± 0.99a | 16.76 ± 1.33b |
| 60 | 18.04 ± 1.93a | 17.48 ± 1.89a | 19.52 ± 2.26b |

Average values with different letter in the same row are statistically significantly different (P < 0.05)

TABLE 4
Percentage survival of L. vannamei after 60 days of
feeding with Aquanin plus at 0, 0.05% and 0.1%.

| Feeding | Percentage survival (%) | | |
|---|---|---|---|
| period | | 0.05% Aquanin | 0.1% Aquanin |
| (day) | control | plus | plus |
| 0 | 100.00 ± 0.00a | 100.00 ± 0.00a | 100.00 ± 0.00a |
| 20 | 93.60 ± 1.35a | 98.68 ± 0.37b | 99.20 ± 0.31b |
| 40 | 83.20 ± 0.11a | 97.18 ± 1.66b | 97.60 ± 0.15b |
| 60 | 74.40 ± 6.07a | 88.00 ± 6.32b | 95.23 ± 1.79b |

Average values with different letter in the same row are statistically significantly different (P < 0.05)

### Experiment 3

Litopenaeus vannamei (11-12 g) were obtained from a commercial shrimp farm in Chantaburi province, Thailand. A total of 1,500 farm-reared shrimp were transported and acclimated in fiberglass tanks. After 14 days of acclimatization, shrimp were used for the experiment. Salinity during the acclimation period and experiment was maintained at 25 ppt.

Three treatments consisted of 0.05% Aquanin Plus, 0.1% Aquanin Plus and control feed were used in the experiment. A total of 150 shrimp for each treatment (and six replications/treatment) were used. The immune parameters of total hemocytes, percentage phagocytosis, phenoloxidase and bactericidal activity were measured every 10 days for 50 days.

### Immune Parameters

### Preparation of Hemolymph Samples

Blood sample of 0.5 ml from each shrimp were withdrawn from the base of 3^{rd} walking leg by a syringe containing 1.5 ml anticoagulant (K-199+5% L-cysteine).

1. Total Hemocytes

After collected hemolymph, hemocytes were counted using a hemocytometer an calculated as the number of blood cells (total hemocytes per cubic millimeter).

2. Phagocytosis

This method was modified from Itami et al. (Enhancement of disease resistance of kuruma prawn Penaeus japonicus and increase in phagocytic activity of prawn hemocytes after oral administration of β-1,3-glucan (Schizophyllan). In Proceeding of the Third Asian Fisheries Forum Singapore 26-30 Oct. 1992. The Asian Fisheries Society Manila, Phillippines. 1994)

Two hundred microliters of hemolymph was collected from the base of 3rd walking leg and mixed with 800 pl of sterile anticoagulant. Collected shrimp hemocytes were rinsed with shrimp saline and the viable cell number adjusted to 1×106 cells/ml. The cell suspension

(200 μl) was inoculated into a cover slip. After 20 minutes, the cell suspension was removed, and rinsed with shrimp saline three times. Heat-killed yeast (2) was added and incubation for 2 hours. After the incubation, heat-killed yeast was removed, rinsed with shrimp saline five times, and fixed with 100% methanol. Then, this cover slip was stained with Giemsa stain and mounted with permount.

Two hundred hemocytes were counted. Phagocytic activity, defined as percentage phagocytosis was expressed as:

$$percentage phagocytosis = \frac{phagocytic hemocytes \times 100}{total hemocytes}$$

3. Phenoloxidase Activity Assay

The method was modified from Supamattaya et al. (The effect of β-glucan (Macrogard®) on growth performance, immune response and disease resistance in black tiger shrimp, Penaeus monodon Fabricus. Songklanakarin J. Sci. Technol. 22:677-688. 2000). After the blood was withdrawn, the hemocytes were washed three times with shrimp saline (1.000 rpm 4° C. 10 min). Haemocyte lysate (HLS) was prepared from hemocytes in a cacodylate buffer pH 7.4 by using the sonicator at 30 amplitute for 5 seconds and the suspension was then centrifuged at 10,000 rpm., 4° C. for 20 min. The supernatant was collected as HLS. Then 200 pl of 0.1% trypsin in cacodylate buffer was mixed to the 200 ml HLS followed by 200 pl of L-dihydroxyphenylalanine (L-DOPA) at 4 mg/ml as the substrate. Enzyme activity was measured as the absorbance of dopachrome at 490 nm. wavelength. Measurement of protein content in HLS was made by using the method of Lowry et al. (Protein measurement with the folin phenol reagent. J. Biol. Chem. 193:265-275. 1951). The phenoloxidase activity was calculated as the increasing of optimum density (OD) per minute per mg of protein as:

1 unit of phenoloxidase=ΔOD490/min/mg protein

4. Bactericidal Activity

Serum was separated from the blood of each shrimp sample and diluted by 2.6% NaCl at 1:2, 1:4, 1:8, 1:16 and 1:32. Then 0.5 ml of each serum dilution and 0.5 ml of NaCl as the control were used in the study. V. harveyi suspension of 0.5 ml (prepared according to the method in 3) was put into each serum dilution and the control. The treatments were incubated at room temperature for 3 h before enumerating the number of bacteria by a spread plate technique. The results were recorded from the dilution that could decrease 50% V. harveyi compared with the control.

The data were subjected to one-way analysis of variance followed by Duncan's multiple range test. Differences were considered significant if P<0.05.

### Results—Determination of Aquanin Plus Effect on Immune Characteristics of White Shrimp

The immune responses were measured by total hemocytes count (THC), percentage phagocytosis, phenoloxidase activity and bactericidal activity. During the experiments, water quality parameters were maintained as follows: temperature at 28±10 C., pH at 7.8-8.0 and salinity at 25 ppt.

### Total Hemocytes Count

After 20 days of feeding, shrimp fed with diets containing 0.1% Aquanin plus had a total hemocytes count of 1.39±0.076×107 cells/ml which was significantly higher (P<0.05) than that of shrimp fed with diets containing 0.05% Aquanin plus and the controls, which had total hemocytes count 8.5±0.41 and 8.9±0.23 cells/ml, respectively. However, after 30-50 days of feeding, both Aquanin plus-treated groups had total hemocytes count significantly higher (P<0.05) than those of the control group (Table 5).

TABLE 5
Total hemocyte count (THC) of L. vannamei after 0, 10, 20, 30, 40
and 50 days of feeding with Aquanin plus at 0, 0.05% and 0.1%.

| THC | | Aquanin plus | |
|---|---|---|---|
| (×106 cells/ml) | Control | 0.05% | 0.1% |
| 0 day | 7.05 ± 0.67a | 7.33 ± 0.59a | 7.65 ± 0.16a |
| 10 day | 8.28 ± 3.43a | 8.30 ± 0.86a | 13.50 ± 5.06a |
| 20 day | 8.50 ± 0.41a | 8.90 ± 0.23a | 13.90 ± 0.76b |
| 30 day | 11.00 ± 0.48a | 12.20 ± 0.44b | 14.95 ± 0.52c |
| 40 day | 11.00 ± 0.53a | 12.70 ± 0.53b | 15.65 ± 0.99c |
| 50 day | 11.25 ± 0.51a | 13.38 ± 0.29b | 15.98 ± 0.29c |

Average values with different letter in the same row are statistically significantly different (P < 0.05)

### Phagocytic Activity

After 20 days of feeding, shrimp fed with diets containing 0.1% Aquanin plus had percentage of phagocytosis significantly higher (P<0.05) than those of the 0.05% Aquanin plus and control groups, which had percentage of phagocytosis 24.72±0.62, 23.79±0.36 and 22.11±0.74, respectively (Table 6).

TABLE 6
Percentage phagocytosis of L. vannamei after 0, 10, 20, 30, 40
and 50 days of feeding with Aquanin plus at 0, 0.05% and 0.1%.

| Percentage | | Aquanin plus | |
|---|---|---|---|
| phagocytosis | Control | 0.05% | 0.1% |
| 0 day | 22.62 ± 0.69a | 21.74 ± 2.50a | 19.88 ± 1.67a |
| 10 day | 22.42 ± 1.75a | 23.68 ± 0.89a | 23.17 ± 1.18a |
| 20 day | 22.11 ± 0.74a | 23.79 ± 0.36ab | 24.72 ± 0.62b |
| 30 day | 22.39 ± 0.57a | 24.24 ± 0.41b | 26.31 ± 0.45c |
| 40 day | 23.51 ± 1.20a | 25.73 ± 0.95ab | 30.73 ± 2.19b |
| 50 day | 25.75 ± 1.34a | 27.86 ± 1.64a | 33.61 ± 0.85b |

Average values with different letter in the same row are statistically significantly different (P < 0.05)

### Phenoloxidase Activity

After 10 days of feeding, shrimp fed with diets containing 0.1% Aquanin plus had phenoloxidase activity of 342.82±109.17 unit/min/mg. protein, which were significantly higher (P<0.05) than that of shrimp fed with diets containing 0.05% Aquanin plus and the controls, which had phenoloxidase activity of 315.11±19.90 and 279.27±81.25 unit/min/mg. protein., respectively (Table 7).

TABLE 7
Phenoloxidase activity of L. vannamei after 0, 10, 20, 30, 40
and 50 days of feeding with Aquanin plus at 0, 0.05% and 0.1%.

| Phenoloxidase activity | | Aquanin plus | |
|---|---|---|---|
| (unit/min/mg. protein) | Control | 0.05% | 0.1% |
| 0 day | 255.84 ± 20.53a | 246.80 ± 17.20a | 251.74 ± 26.18a |
| 10 day | 279.27 ± 81.25a | 315.11 ± 19.90a | 342.82 ± 109.17b |
| 20 day | 282.03 ± 26.91a | 318.73 ± 14.61a | 406.67 ± 26.53b |
| 30 day | 290.95 ± 28.83a | 301.42 ± 14.41ab | 426.37 ± 43.18b |
| 40 day | 303.85 ± 25.37a | 377.93 ± 39.86ab | 471.47 ± 46.82b |
| 50 day | 307.08 ± 18.92a | 326.65 ± 19.98a | 441.98 ± 32.63b |

Average values with different letter in the same row are statistically significantly different (P < 0.05)

### Bactericidal Activity

Shrimp fed with 0.05% and 0.1% Aquanin plus had bactericidal activity at the serum dilution of 1:16 while control were 1:8 after 10 days of experiment (Table 8).

TABLE 8
Bactericidal activity of L. vannamei after 0, 10, 20, 30, 40 and
50 days of feeding with Aquanin plus at 0, 0.05% and 0.1%.

Aquanin plus

| Bactericidal activity | Control | 0.05% | 0.1% |
|---|---|---|---|
| 0 day | 1:8 | 1:8 | 1:8 |
| 10 day | 1:8 | 1:16 | 1:16 |
| 20 day | 1:8 | 1:16 | 1:16 |
| 30 day | 1:8 | 1:16 | 1:16 |
| 40 day | 1:8 | 1:16 | 1:16 |
| 50 day | 1:8 | 1:16 | 1:16 |

### Experiment 4

Litopenaeus vannamei (11-12 g) were obtained from a commercial shrimp farm in Chantaburi province, Thailand. A total of 1,500 farm-reared shrimp were transported and acclimated in fiberglass tanks at Aquaculture Business Research Unit laboratory, Faculty of Fisheries, Kasetsart University. After 14 days of acclimatization, shrimp were used for the experiment. Salinity during the acclimation period and experiment was maintained at 25 ppt.

After 60 days of feeding, shrimp from experiment 1 (60 shrimp/treatment) were challenged with virulent strain of V. harveyi which had been cultured in Tryptic Soy Agar (TSA) with 1.5% NaCl (w/v). All shrimp were injected intramuscularly by 0.1 ml of V. harveyi suspension at 2×105 CFU/ml (48 h LD50). Cumulative mortality of shrimp from all treatment groups were recorded for 7 days.

The data were subjected to one-way analysis of variance followed by Duncan's multiple range test. Differences were considered significant if P<0.05.

### Results—Effect of Aquanin Plus White Shrimp Survival Following Infection by Vibrio Harveyi and White Spot Syndrome Virus (WSSV)

After 7 days post intramuscular injection with V. harveyi 2×10⁵ CFU/ml. in shrimp fed with different levels of Aquanin plus (0, 0.05 and 0.1%), both Aquanin plus groups had 100% survival while in the control group the survival rate was 56.67% (Table 9).

TABLE 9
Percentage mortality of L. vannamei after 60 days of feeding with
Aquanin plus at 0, 0.05% and 0.1% and infected by Vibrio harveyi.

| | % mortality | | |
|---|---|---|---|
| Day | Control | Aquanin plus 0.05% | Aquanin plus 0.1% |
| 0 | 0.00 | 0.00 | 0.00 |
| 1 | 13.33 | 0.00 | 0.00 |
| 2 | 43.33 | 0.00 | 0.00 |
| 3 | 43.33 | 0.00 | 0.00 |
| 4 | 43.33 | 0.00 | 0.00 |
| 5 | 43.33 | 0.00 | 0.00 |
| 6 | 43.33 | 0.00 | 0.00 |
| 7 | 43.33 | 0.00 | 0.00 |

Mortality of shrimp from all Aquanin plus fed with infected-WSSV shrimp was shown in Table 10. In control group mortality was 100% within 6 days while for both Aquanin plus groups 100% mortality was observed at day 7.

TABLE 10
Percentage mortality of L. vannamei after 60 days
of feeding with Aquanin plus at 0, 0.05% and 0.1%
and infected by white spot syndrome virus.

| | % mortality | | |
|---|---|---|---|
| Day | Control | Aquanin plus 0.05% | Aquanin plus 0.1% |
| 0 | 0.00 | 0.00 | 0.00 |
| 1 | 10.00 | 0.00 | 0.00 |
| 2 | 40.00 | 6.67 | 0.00 |
| 3 | 50.00 | 23.33 | 0.00 |
| 4 | 63.33 | 46.67 | 33.33 |
| 5 | 80.00 | 60.00 | 46.67 |
| 6 | 100.00 | 96.67 | 80.00 |
| 7 | 100.00 | 100.00 | 100.00 |

After challenged shrimp were fed with Aquanin plus at 3 concentrations (0, 0.05 and 0.1%) for 60 days with yellow head virus, 100% mortality was observed at day 6 in the control group, all shrimp in the control group died in 6 days. In contrast, mortality was observed in the Aquanin plus groups at day 7 (Table 11).

TABLE 11
Percentage mortality of L. vannamei after 60
days of feeding with Aquanin plus 0, 0.05% and
0.1% and infected by yellow head virus.

| | % mortality | | |
|---|---|---|---|
| Day | Control | Aquanin plus 0.05% | Aquanin plus 0.1% |
| 0 | 0.00 | 0.00 | 0.00 |
| 1 | 0.00 | 0.00 | 0.00 |
| 2 | 0.00 | 0.00 | 0.00 |
| 3 | 45.00 | 15.00 | 0.00 |
| 4 | 75.00 | 45.00 | 25.00 |
| 5 | 95.00 | 70.00 | 55.00 |
| 6 | 0.00 | 0.00 | 80.00 |
| 7 | 0.00 | 0.00 | 0.00 |

Experiments 2-4 above demonstrate that dietary Aquanin plus at 0.1% has growth-promoting effects on L. vannamei, evaluated by measuring the increases in body weight. This growth-promoting effect may be due to β-cyclodextrin cysteamine hydrochloride, which is present at about 27% in Aquanin plus. Cysteamine is known to produce a direct or indirect increases in Growth Hormone (GH) release and growth rates in mammals and chickens (Hall et al., Control of growth hormone secretion in the vertebrates: a comparative survey. Comp. Biochem. Physiol. 84:231-253. 1986) but has yet to be analyzed with respect to shellfish. In grass carp (C. idellus), cysteamine hydrochloride was applied to promote cultured fish growth in intensive large-scale aquaculture (Xia and Lin, Cysteamine—a somatostatin-inhibiting agent-induced growth hormone secretion and growth acceleration in juvenile grass carp (Ctenopharyngodon idellus). Gen. Comp. Endocrinol. 134:285-295. 2003). Cysteamine and cysteamine hydrochloride have many advantages such as absence of species specificity, simple chemical composition, convenient dietary administration of the drugs to farmed fish and low cost (Xia and Lin, 2003).

Considering Aquanin plus' effect on immune characteristics, the results from these experiments suggest that the application of Aquanin plus at 0.1% effectively enhanced the total hemocytes and percentage phagocytosis of L. vannamei in 20 days and enhanced the phenoloxidase activity and bactericidal activity after 10 days. Further, use of 0.05% of Aquanin plus increased total hemocytes and bactericidal activity. Without being bound to any theory, it appears that cysteamine and vitamin F (which may enhance cellular and humoral immune response) of Aquanin plus may play an important part in enhancing the immunostimulant ability for L. vannamei culture. In conclusion, the results from these experiments suggest that, with regard to L. vannamei diet, Aquanin plus at 0.1% effectively enhances the growth, survival rate and the immune response (including THC, percentage phagocytosis and phenoloxidase activity and bactericidal activity) after 40 days of feeding.

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

I claim:

1. A composition comprising cysteamine, or a salt thereof, a carrier, shellfish feed and vitamin E, wherein the cysteamine, or salt thereof, is present at about 0.01% to 10% of the composition.

2. The composition of claim 1, wherein the cysteamine is cysteamine hydrochloride.

3. The composition of claim 1, wherein the cysteamine is cysteamine hydrochloride and wherein the composition comprises about 250-500 g of cysteamine hydrochloride per ton of the composition.

4. The composition of claim 1, comprising β-cyclodextrin.

5. A method of improving the immunity in pacific white shrimp (Litopenaeus vannamei) against infection, wherein the method comprises administering to the shrimp a composition comprising an effective amount of cysteamine or a salt thereof.

6. The method of claim 5, wherein the infection is selected from the group consisting of White Spot Syndrome Baculovirus Complex of Penaeid Shrimp; Yellow-head Virus Disease (YHD) of Penaeid Shrimp; and spp. (Vibrio Disease) of cultured shrimp.

7. The method of claim 5, wherein the composition comprises cysteamine hydrochloride.

8. The method of claim 7, wherein the dosage of cysteamine hydrochloride administered is between about 1-20 μg cysteamine hydrochloride per gram of shrimp per day.

9. The method of claim 7, wherein the dosage of cysteamine hydrochloride administered is about 3-15 μg cysteamine hydrochloride per gram of shrimp per day.

10. The method of claim 5, wherein the composition further comprises β-cyclodextrin.

11. The method of claim 5, wherein the composition comprises Aquanin plus.

12. The method of claim 11, wherein the composition comprises at least 0.05% Aquanin plus.

13. The method of claim 12, wherein the composition comprises at least 0.1% Aquanin plus.
